(19) United States
(12) Patent Application Publication
(10) Pub. No.: US 2003/0181774 A1
(43) Pub. Date: Sep. 25, 2003

(54) Process for the transalkylation of benzene and C9+ aromatics

(75) Inventors: Dejin Kong, Shanghai (CN); Yu Xing, Shanghai (CN)

Correspondence Address: COHEN, PONTANI, LIEBERMAN & PAVANE, 551 FIFTH AVENUE, SUITE 1210, NEW YORK, NY, 10176, US

(73) Assignee: China Petroleum & Chemical Corporation & Shanghai Research Institute of Petrochemical Technology

(21) Appl. No.: 10/385,988

(22) Filed: Mar. 11, 2003

(30) Foreign Application Priority Data
Mar. 13, 2002 (CN) 02111004.2
Mar. 13, 2002 (CN) 02111005.0

(51) Int. Cl. C07C 5/10

(52) U.S. Cl. 585/475

(57) ABSTRACT

A transalkylation process of benzene and C₉⁺ aromatics for the production of toluene and C₈ aromatics. The transalkylation is operated in the presence of hydrogen, at a benzene to C₉⁺A ratio of 10-90:90-10 by weight, a reaction temperature of 300-600□, a reaction pressure of 1.0-6.0 MPa, a weight space velocity of the feed stream of 0.5-5.0 h⁻¹, and a hydrogen to hydrocarbon mole ratio of 1-15 in a gas-solid phase fixed bed reactor having a transalkylation catalyst comprising H-zeolite and molybdenum.

### FIELD OF THE INVENTION

The present invention relates to a process for the transalkylation of benzene (Ben) and C₉ and more than C₉ aromatics (C₉⁺A). In particular it relates to a process for producing toluene (Tol) and C₈ aromatics (C₈A) to be provided for the selective toluene proportionation unit and C₈A isomerization unit to increase the output of p-xylene through the transalkylation reaction of benzene (Ben) and C₉⁺ aromatics.

### BACKGROUND OF THE INVENTION

p-xylene is one of the major basic organic feedstocks in petrochemical industry and has widespread applications in many fields such as chemical fiber, synthetic resin, pesticide, medicine, plastic, etc. The typical process for producing p-xylene (PX) is to separate p-xylene in the ethylbenzene-containing xylenes, i.e. C₈ aromatics in thermodynamic equilibrium produced in the catalytic reforming of naphtha from its isomer mixture with near boiling points through a multi-stage subzero crystallization separation technique or molecular sieve simulation moving bed separation (abbreviated as adsorptive separation) technique. As for the treatment of o-xylene and m-xylene, C₈A isomerization (abbreviated as isomerization) technique is generally used to isomerize them to p-xylene. Utilization of disproportionation of toluene, or disproportionation and transalkylation of toluene and C₉⁺ aromatics (C₉⁺A) to produce benzene and C₈A, and thereby increase the output of C₈A is an effective route for increasing the output of p-xylene.

So far, the more typical and mature processes relating to toluene disproportionation in the world are the Tatoray traditional toluene disproportionation process industrialized in the end of 1960s, the MTDP process developed in the end of 1980s, and the S-TDT process and TransPlus process developed in recent years. The selective disproportionation of toluene is a new route for producing p-xylene. Since the selective disproportionation of toluene on modified ZSM-5 catalysts can produce benzene and C₈A with a high concentration of p-xylene, only one simple subzero crystallization separation step is needed to separate a large part of high purity p-xylene. In recent years, along with the enhancement of the catalyst performance, this process has made a great progress. The typical processes are the MSTDP toluene disproportionation process industrialized late 1980s and PX-Plus process developed in recent years.

In the industrializedselective toluene disproportionation process-MSTDP, a treated ZSM-5 mesoporous molecular sieve is used as the catalyst to treat a toluene feedstock, yielding C₈A with a high concentration of p-xylene (85-90% by weight, the same bellow except otherwise noted) and nitration grade benzene. In the PX-Plus process, the report on the industrialization of which has not seen, the major process indices are a PX selectivity of 90%, a mole ratio of benzene to PX of 1.37 in case of the toluene conversion being 30%.

However, in this kind of processes for selective toluene disproportionation, a high para selectivity is accompanied by a harsh requirement for the feedstock selection, and only toluene can be used as the feedstock. C₉⁺A has no use in this kind of processes, and at least it cannot be directly used. Besides, the process also produces a great amount of benzene, resulting in a low yield of p-xylene. This is a vital shortcoming of the process for selective disproportionation.

The feeds for the reactor of a typical Tatoray Process are toluene and C₉ aromatics (C₉A), and the content of C₁₀⁺ aromatics (C₁₀ and higher aromatics) must be strictly controlled. The literature based on the Tatoray Process includes U.S. Pat. No. 4,341,914, CN98110859.8, U.S. Pat. No. 2,795,629, U.S. Pat. No. 3,551,510, CN97106719.8, etc. In order to enhance the economic benefits of the device, the process flow of the Tatoray Process has been further studied and optimized, focusing on the kernel technique, preparation of the catalyst, to improve its overall performance and indices, such as raising the weight hourly space velocity, prolonging the operation cycle and increasing the average molecular weight of the aromatics feedstocks. The increase in the average molecular weight benefits the increase in C₈A yield, but in order to maintain a certain conversion, i.e. maintain a certain activity of the catalyst, too high a content of heavy aromatics would certainly lead to the intensifying of side reactions especially the hydrodealkylation reaction, thereby lead to the increase of the benzene in the reaction product, decrease of C₈A/Ben ratio, great lose of aromatics, obtaining less C₈A and more Ben when treating the same feedstock. As for an aromatics complex, the reason why the toluene disproportionation device cannot be lacked is that it has the function of providing C₈A. The increase in the amount of Ben and decrease in the amount of C₈A are obviously unfavorable to the whole aromatics complex. These shortcomings have limited the development of this kind of process.

It is readily seen from summarizing the above processes that all these patents are formed by making reasonable modification in some one or more aspects for a certain particular catalyst for toluene disproportionation and transalkylation such as the schemes of the transalkylation ability of heavy aromatics and the separation of the reaction products, but the limit to the original process idea has not been broken through. Their common shortcomings are that when toluene or toluene and C₉⁺A are used to produce C₈A and thereby increase the output of p-xylene, benzene is inevitably produced as a by-product, and the heavy aromatics cannot be fully utilized.

The traditional aromatics transalkylation catalyst and process mainly produce benzene and xylene with toluene and C₉⁺A as the feedstocks, but the great amount of benzene produced is usually an unsalable product.

The catalyst and process for toluene shape-selecting disproportionation possess a competitive power only in a limited scope because they cannot utilize C₉⁺A.

The hydrodealkylation process can produce benzene, toluene, and xylene, but also can produce polycyclic or condensed nuclear compounds through side reactions such as the condensation reaction of aromatics, etc, and the higher the temperature, the more intense the side reaction becomes, the more large molecule condensed product forms, the more coke deposits on the catalyst, and the faster the catalyst deactivates.

The environmental legislations become stricter and stricter in various countries, especially the content of aromatics in gasoline has to gradually decrease. Thus, the petroleum companies of various countries have to consider the extraction of the excessive benzene to accord with the environmental legislation. Along with the extension and reconstruction of the ethylene device, as a by-product, benzene will become more and more. Although benzene occupies a decisive position in the petrochemical sector since it is a basic feedstock for synthesizing styrene, phenols, and anhydrous maleic acid, etc, the demand for benzene is difficult to increase in these fields. Therefore, there is a necessity to develop a new market and find new application areas for benzene.

Presently, the literature relating to the transalkylation reaction between benzene and C₉⁺A is very little. JP10158201 discloses a process, the object of which is to produce polyalkylbenzene through the transalkylation between benzene or toluene and polyalkyl aromatic compounds. The catalyst used in this patent is a H-DGA zeolite, which is unmodified with metals, and there is no report on the metal modification and stability of the catalyst.

### SUMMARY OF THE INVENTION

The technical problem to be solved by the present invention is to overcome the problem existing in the aforesaid literature that the process for the transalkylation of benzene and C₉⁺A or the possibility of converting C₁₀⁺A to useful toluene and C₈A have not been involved in the literature, to provide a process for producing toluene and co-producing a small amount of C₈A with benzene and C₉⁺A as feedstocks, and thereby providing toluene for the selective disproportionation unit and providing C₈A for the aromatics isomerization unit, and make full use of benzene and C₉⁺A as reaction feedstocks to increase the output of p-xylene through the combined action of the transalkylation of aromatics, selective disproportionation of toluene and isomerization of aromatics. Besides, the process provided by the present invention for the transalkylation of benzene and C₉⁺A has the characters of adaptability to broad process conditions, high activity and good stability of the catalyst, and high selectivity to the target product.

To solve the above technical problem, the present invention adopts the following technical solution: a process for the transalkylation of benzene and C₉⁺A, which uses benzene and C₉⁺A as the reaction feedstocks to conduct the transalkylation reaction, yielding toluene and C₈A, which comprises:

contacting the benzene and C₉⁺ aromatics with a catalyst by passing through a gas-solid phase fixed bed reactor in the presence of hydrogen under the conditions of a weight ratio of benzene to C₉⁺ aromatics of 10-90:90-10, a reaction temperature of 300-600° C., a reaction pressure of 1.0-6.0 MPa, a weight hourly space velocity WHSV of the aromatics feed of 0.5-5.0 h⁻¹, and a hydrogen to hydrocarbon mole ratio of 1- 15. The used catalyst contains 10-90 parts by weight of a H-zeolite, wherein the SiO₂/Al₂O₃ mole ratio of the H-zeolite is 3-500, 0.01-20 parts by weight of the molybdenum metal or/and oxide thereof supported on the H-zeolite and 10-90 parts by weight of the alumina binder based on the weight of the catalyst.

### DETAILED DESCRIPTION OF THE PRESENT INVENTION

In the above technical solution, the SiO₂/Al₂O₃ mole ratio in the H-zeolite is preferably in the range of 15-100, more preferably 15-50. The zeolite is preferably at least one selected from the group consisting of ZSM-5 zeolite, Y-zeolite, mordenite, β-zeolite, MCM-22 zeolite, MCM-49 zeolite, or MCM-56 zeolite, with mordenite or/and β-zeolite being more preferable. The constraint factor of the zeolite is in the range of 0.1-15. The amount of molybdenum metal and/or oxide thereof is preferably in the range of 0.1-10.0 parts by weight, more preferably 0.1-4 parts by weight. 0.01-20 parts by weight of at least one metal or/and oxide thereof selected from the group consisting of iron, cobalt, nickel, chromium, tungsten, bismuth, lanthanum, zirconium, or silver is also supported on the zeolite, and the preferred range of the amount is 0.1-5 parts by weight. The metal or oxide thereof supported on the zeolite is preferably at least one selected from the group consisting of cobalt, chromium, or bismuth. The H-zeolite may be directly synthesized one or a dealuminized one. The constraint factor and its detecting method are described in U.S. Pat. No. 4,016,218 in detail. The preferred C₉⁺A in the feedstock are at least one selected from the group consisting of trimethylbenzene, methylethylbenzene, propylbenzene, indan, C₁₀⁺ heavy aromatics, and the preferred C₉A are xylenes, ethylbenzene or a mixture thereof.

Since the H-zeolite in the catalyst used in the present invention is modified with metals, the ability of the catalyst to resist water in the feedstock is greatly enhanced, and a high activity and stability can be maintained, i.e., the feedstock containing 500 ppm of water can be used and the catalyst can be used under rigorous conditions. For industrial devices, the dehydration operation of the feedstock of Ben and C₉⁺ can be avoided.

The major components of the catalyst used in the present invention is 0.01-20 parts of molybdenum metal or/and oxide thereof and optionally 0.01-20 parts of at least one metal or/and oxide thereof selected from the group consisting of iron, cobalt, nickel, chromium, tungsten, bismuth, lanthanum, zirconium, or silver supported on the H-zeolite. The H-zeolite can be either a dealuminized zeolite derived by treating a zeolite of a low SiO₂/Al₂O₃ ratio with an acid, or a natural zeolite or a high silica H-zeolite derived by inorganic ion exchanging, drying and calicining a high silica Na-zeolite, which is yielded by the direct crystallization. The content of sodium in the zeolite should be lower than 0.2% by weight, and the high silica zeolite synthesized by direct crystallization is most suitable. The excellent performance of the catalyst of the present invention is a result of the co-action of the catalysis of the high silica H-zeolite and the catalysis of the metal and/or metal oxide supported on the zeolite.

The present invention process prepares the catalyst for the transalkylation of Ben and C₉⁺A, using an ordinary solution impregnation process followed by drying and calcinations. This process possesses the characters of mature process, simple equipment, convenient operation, and ease to be industrialized.

When the invention of the present invention is used in the transalkylation process of benzene and C₉⁺A, a better technical effect is achieved, e.g., the selectivity to toluene plus C₈A attains 94.1% at most, and the conversion of benzene plus C₉⁺A attains 64% at most, the stability is good, high contents of water and C₁₀⁺A are permitted, and possibility to convert to useful benzene and C₈ aromatics.

The data on the transalkylation reaction of benzene and C₉⁺A are processed with the following formulas:

$$\begin{matrix}Conversion of benzene = \frac{{Ben}_{i n} - {Ben}_{ou t}}{{Ben}_{i n}} \times 100 \% ( weight ) \\ Conversion of {C}_{9}^{+} A = \frac{{C}_{9}^{+} {A}_{i n} - {C}_{9}^{+} {A}_{out}}{{C}_{9}^{+} {A}_{i n}} \times 100 \% ( weight ) \\ Total conversion = \frac{{( Ben + {C}_{9}^{+} A )}_{i n} - {( Ben + {C}_{9}^{+} A )}_{out}}{{( Ben + {C}_{9}^{+} A )}_{i n}} \times 100 \% ( weight ) \\ Selectivity to toluene + {C}_{8} A = \frac{yielded ( toluene + {C}_{8} A )}{Reacted ( Ben + {C}_{9}^{+} A )} \times 100 \% ( weight )\end{matrix}$$

The present invention will be further described by the examples below.

### EXAMPLES

### Example 1-3

Na-mordenite, Na-β-zeolite, and Na-ZSM-5 zeolite were prepared according to CN89106793.0, U.S. Pat. Nos. 3308069, and 4,441,991 respectively. The three Na-zeolites were ion-exchanged with the aqueous solution of ammonium chloride or ammonium nitrate at 90-98° C. for 1-8 h. The mother liquid was removed by filtration and the ion-exchange was repeated several times, and then the zeolite was washed, dried at 110° C., yielding ammonium-zeolite. The SiO₂/Al₂O₃ ratios of the three ammonium-zeolites were 25, 25 and 500 respectively. The three ammonium-zeolites were mixed with pseudo-boehmite (α-Al₂O₃.H₂O) respectively, and dilute nitric acid, ammonium molybdate, and water were added to the mixture, which was kneaded to become uniform, extruded, dried at 110° C., pelleted, and calcined at 550° C., yielding Catalysts A1, A2, and A3 with a same molybdenum content of 4.0% by weight.

### Example 4

A commercial Na-Y-zeolite (SiO₂/Al₂O₃=4) was allowed to conduct ion-exchange, kneading, shaping, and calcinations according to the process in Example 1-3, yielding Catalyst A4, wherein the content of metal molybdenum was 4.0% by weight.

### Examples 5-7

Na-MCM-22 zeolite, Na-MCM-49 zeolite, and Na-MCM-56 zeolite were prepared according to U.S. Pat. Nos. 4,956,514, 5,264,643, and 5,453,554 respectively. The three Na-zeolites were ion-exchanged with the aqueous solution of ammonium chloride or ammonium nitrate at 90-98° C. for 1-8 h. The mother liquid was removed by filtration and the ion-exchange was repeated several times, and then the zeolites were washed, dried at 110° C., yielding ammonium-zeolite. The SiO₂/Al₂O₃ ratios of the three ammonium-zeolites were 30, 138 and 18 respectively. The three ammonium-zeolites were mixed with pseudo-boehmite (α-Al₂O₃.H₂O) respectively, and dilute nitric acid, ammonium molybdate, and water were added to the mixture, which was kneaded to become uniform, kneaded, dried at 110° C., extruded, and calcined at 550° C., yielding Catalysts A5, A6, and A7 with a same molybdenum content of 4.0% by weight.

### Examples 8-13

Four β-zeolites with SiO₂/Al₂O₃ ratios of 25.7, 35.1, 40.2, and 44.0 were prepared respectively according to U.S. Pat. No. 3,308,069. After passing through ammonium-exchange and drying, the β-zeolites were mixed with commercial pseudo-boehmite (α-Al₂O₃.H₂O) in a ratio of 50/50 (weight) respectively, and then dilute nitric acid and water were added. The mixture was kneaded to become uniform, extruded, dried at 110° C., and calcined at 400° C., yielding cylinders. The cylinders was impregnated in the aqueous solution of ammonium molybdate overnight, and then dried at 110° C., calcined at 540° C., yielding Catalysts B1, B2, B3, and B4 containing 4.0% by weight of molybdenum. Catalysts B5 and B6 were derived with Hβ-zeolite to the commercial pseudo-boehmite weight ratios of 70/30 and 30/70 respectively according to the same process.

### Examples 14-16

The present example was to prepare molybdenum-doped dealuminized mordenite catalysts. The preparation of the dealuminized mordenite: the commercial Na-mordenite (SiO₂/Al₂O₃=10) was fluxed with dilute nitric acid at 90° C. to allow mordenite to dealuminize, and then filtered, washed dried at 110° C., yielding the dealuminized mordenite of SiO₂/Al₂O₃=15.2. The above operation was repeated for several times and dealuminized mordenites with a SiO₂/Al₂O₃ ratio of 20.4 and 26.7 were yielded respectively. The three dealuminized mordenites were mixed with commercial pseudo-boehmite (α-Al₂O₃.H₂O) in a ratio of 50/50 (weight) respectively, and dilute nitric acid, ammonium molybdate, and water were added to the mixture, which was kneaded to become uniform, extruded, dried at 110° C., and calcined at 550° C., yielding Catalysts C1, C2, and C3 with a same molybdenum content of 4.0% by weight.

### Examples 17-19

According to the process of Examples 8-13, the β-zeolite of SiO₂/Al₂O₃=25.7 was kneaded with pseudo-boehmite and the mixture was shaped by extrusion and impregnated with the aqueous solution of ammonium molybdate, yielding Catalysts D1, D2, D3, D4, and D5 with the molybdenum contents of 0.05%, 1.0%, 3.0%, 8.0%, and 16.0% by weight respectively.

### Example 20-22

The ammonium-mordenite in Examples 1-3 was mixed with commercial pseudo-boehmite (α-Al₂O₃.H₂O) in different ratios, and dilute nitric acid and water were added. The mixture was kneaded to become uniform, dried at 110° C., extruded and calcined at 400° C., yielding cylinders. The cylinders was impregnated in the aqueous solution of ammonium molybdate overnight, then dried at 110° C., calcined again at 540° C., yielding Catalysts E1, E2, E3, E4, and E5 containing 0.05%, 1.0%, 3.0%, 8.0%, and 16%

### Comparative Example 1

HM zeolite catalyst F1 containing 0.0% by weight of molybdenum metal was derived according to the process of Examples 1-3.

### Comparative Example 2

Hβ zeolite catalyst F2 containing 0.0% by weight of molybdenum metal was derived according to the process of Examples 13-15.

### Example 23

The activities of various catalysts prepared in Examples 1-22 and Comparative Examples 1-2 for the transalkylation reaction were evaluated in a pressurized fixed-bed reactor. The loading of the catalyst was 20 g, reaction temperature 380° C., reaction pressure 3.0 MPa (gauge pressure), hydrogen/hydrocarbon mole ratio 5.3, the Ben/C₉A/C₁₀A⁺ weight ratio in the feedstock 57/43/5, water content 500 ppm, weight hourly space velocity WHSV 2.5 h⁻¹, and the results are shown in Table 1. The results show that the molybdenum-supported H-zeolite catalyst prepared by the present invention possesses excellent activity for the transalkylation reaction.

### Examples 24-28

The ammonium-mordenite in Examples 1-3 was mixed with pseudo-boehmite (α-Al₂O₃.H₂O) in a ratio of 70/30, and dilute nitric acid and water were added. The mixture was kneaded to become uniform, extruded, dried at 110° C., impregnated with the aqueous solution of ammonium molybdate and the aqueous solution of cobalt nitrate, dried at 110° C., calcined at 550° C., yielding Catalyst G1 containing 4.0%, and 0.6% by weight of molybdenum metal and cobalt metal respectively. Catalysts G2-G8 containing molybdenum metal and at least one metal or oxide thereof selected from the group consisting of cobalt, nickel, chromium, tungsten, bismuth, lanthanum, zirconium, or silver were yielded in the similar method with G1.

### Example 29

The activities of catalysts G1-G8 prepared in Examples 24-28 for transalkylation were examined using the activity evaluation device and conditions in Example 23 and the results are shown in Table 2. The results show that the catalyst containing molybdenum and at least one metal or oxide thereof selected from the group consisting of cobalt, nickel, chromium, tungsten, bismuth, lanthanum, zirconium, or silver prepared by the present invention possesses excellent activity for the transalkylation reaction.

### Example 30

The activity of catalyst G3 prepared in Examples 24-28 for transalkylation was examined using the activity evaluation device and conditions in Example 23 but feedstocks with different compositions, and the results are shown in Table 3. The results show that in case of feedstocks with different compositions the catalyst prepared by the present invention possesses excellent transalkylation activity, selectivity and adaptability to the feedstocks with different compositions.

### Example 31

The activity of catalyst G3 prepared in Example 24-28 for transalkylation was examined using the activity evaluation device in Example 23 but different reaction conditions, and the results are shown in Table 4. The results show that the catalyst prepared by the present invention possesses excellent transalkylation activity, selectivity and adaptability to different process conditions.

### Example 32

The activities of catalysts A1 and B1 prepared in Example 1-3 and Example 8-13 and comparative catalysts F1 and F2 for transalkylation were further examined for their stability. The reaction conditions were basically the same as those of Example 23, but the initiative temperature for Catalysts A1 and B1 was 380° C., while that for Comparative Catalysts F1 and F2 was 410° C. The conversions of Catalysts A1 and B1 were controlled at about 61-63% by weight and maintained by gradually raising the reaction temperature. The conversions of Catalysts F1 and F2 were controlled at about 48-51% by weight and maintained by gradually raising the reaction temperature. The experimental results are shown in Table 5. It can be seen that during the time on stream of 1000 h with catalyst A1 of the present invention, the reaction temperature always remained stable at 380° C., the average conversion was 62.2% by weight, and the average selectivity to (Tol+C₈A) was 93.0% by weight, while during the time on stream of 500 h with comparative catalyst F1, the reaction temperature rose from 410° C. to the final 445° C. to keep an average conversion of 48.0% by weight and an average selectivity to (Tol+C₈A) of 84.5% by weight. During the time on stream of 1000 h with catalyst B1 of the present invention, the reaction temperature rose from 380° C. to the final 392° C., the average conversion was 63.2% by weight, the average selectivity to (Tol+C₈A) was 90.4% by weight; while during the time on stream of 500 h with comparative catalyst F2, the reaction temperature rose from 410° C. to the final 438° C. to keep an average conversion of 50.3% by weight and an average selectivity to (Tol+C₈A) of 82.9% by weight. The experimental results on the catalyst stability show that the catalyst of the present invention possesses a high activity, high selectivity, and good stability.

### Example 33-35

Three mordenites with SiO₂/Al₂O₃ ratios of 10, 20, and 40, were prepared respectively according to CN89106793. After passing through ammonium-exchange and drying, the mordenites were mixed with commercial pseudo-boehmite (α-Al₂O₃.H₂O) in a ratio of 50/50 by weight, and then dilute nitric acid and water were added. The mixture was kneaded to become uniform, extruded, dried at 110° C., and calcined at 400° C., yielding cylinders. The cylinders were impregnated in the aqueous solution of ammonium molybdate overnight, and then dried at 110° C., calcined at 540° C., yielding Catalysts W1, W2, and W3, containing 4.0% by weight of molybdenum.

### Example 36

The activity of Catalysts W1-W3 prepared in Examples 33-35 for transalkylation was examined using the activity evaluation device and conditions in Example 23, and the results are shown in Table 1. The results show that the molybdenum-containing mordenite catalysts with different SiO₂/Al₂O₃ ratios prepared by the present invention possess excellent activity for transalkylation reaction.

TABLE 1
Results on the catalyst evaluation

| Catalyst | | | | | | | | |
|---|---|---|---|---|---|---|---|---|
| | | SiO2/ | | | | | | |
| | | Al2O3 | Zeolite/ | Content | | | | Tol |
| | | mole | alumina | of | Method | Ben + | Ben + | selec./ |
| | Type | ratio | weight | metal | for | C9 + A | C9 + A | C8A |
| | of | of | ratio of | Mo | adding | conv. | selec. | selec. |
| No. | zeolite | zeolite | zeolite | (wt %) | Mo | (wt %) | (wt %) | (mole) |
| A1 | HM | 25 | 50/50 | 4.0 | Knead | 62.5 | 93.1 | 3.4 |
| A2 | Hβ | 25 | 50/50 | 4.0 | Knead | 63.7 | 90.6 | 3.0 |
| A3 | HZSM-5 | 500 | 50/50 | 4.0 | Knead | 42.6 | 74.4 | 0.8 |
| A4 | HY | 4 | 50/50 | 4.0 | Knead | 51.1 | 76.4 | 1.0 |
| A5 | HMCM-22 | 30 | 50/50 | 4.0 | Knead | 64.0 | 91.6 | 3.2 |
| A6 | HMCM-49 | 138 | 50/50 | 4.0 | Knead | 58.2 | 92.6 | 3.3 |
| A7 | HMCM-56 | 18 | 50/50 | 4.0 | Knead | 64.3 | 91.9 | 3.3 |
| B1 | Hβ | 25.7 | 50/50 | 4.0 | Impreg. | 63.3 | 90.7 | 3.0 |
| B2 | Hβ | 35.1 | 50/50 | 4.0 | Impreg. | 63.5 | 90.4 | 2.9 |
| B3 | Hβ | 40.2 | 50/50 | 4.0 | Impreg. | 62.7 | 90.8 | 2.9 |
| B4 | Hβ | 44.0 | 50/50 | 4.0 | Impreg. | 62.6 | 90.8 | 2.9 |
| B5 | Hβ | 25.7 | 70/30 | 4.0 | Impreg. | 64.1 | 90.2 | 3.1 |
| B6 | Hβ | 25.7 | 30/70 | 4.0 | Impreg. | 57.6 | 90.0 | 2.5 |
| C1 | De- | 152 | 50/50 | 4.0 | Knead | 53.6 | 77.6 | 1.0 |
| | aluminized | | | | | | | |
| | MOR* | | | | | | | |
| C2 | De- | 204 | 50/50 | 4.0 | Knead | 53.9 | 80.4 | 1.1 |
| | aluminized | | | | | | | |
| | MOR | | | | | | | |
| C3 | De- | 267 | 50/50 | 4.0 | Knead | 55.7 | 80.8 | 1.3 |
| | aluminized | | | | | | | |
| | MOR | | | | | | | |
| W1 | MOR | 10 | 50/50 | 4.0 | Knead | 58.2 | 91.1 | 3.0 |
| W2 | MOR | 20 | 50/50 | 4.0 | Knead | 62.1 | 93.2 | 3.5 |
| W3 | MOR | 40 | 50/50 | 4.0 | Knead | 57.0 | 93.5 | 3.1 |
| D1 | Hβ | 25.7 | 50/50 | 0.05 | Impreg. | 50.1 | 84.5 | 1.8 |
| D2 | Hβ | 25.7 | 50/50 | 1.0 | Impreg. | 55.0 | 84.6 | 2.3 |
| D3 | Hβ | 25.7 | 50/50 | 3.0 | Impreg. | 60.7 | 88.2 | 2.8 |
| D4 | Hβ | 25.7 | 50/50 | 8.0 | Impreg. | 66.4 | 87.1 | 3.1 |
| D5 | Hβ | 25.7 | 50/50 | 16.0 | Impreg. | 68.6 | 82.3 | 3.5 |
| E1 | HM | 25.2 | 50/50 | 0.05 | Impreg. | 48.8 | 90.5 | 2.4 |
| E2 | HM | 25.2 | 50/50 | 1.0 | Impreg. | 54.3 | 91.4 | 3.0 |
| E3 | HM | 25.2 | 50/50 | 3.0 | Impreg. | 60.4 | 92.9 | 3.2 |
| E4 | HM | 25.2 | 50/50 | 8.0 | Impreg | 65.2 | 90.8 | 3.6 |
| E5 | HM | 25.2 | 50/50 | 16.0 | Impreg | 66.4 | 88.4 | 3.8 |
| F1 | HM | 25.2 | 50/50 | 0.0 | — | 49.0 | 87.7 | 1.3 |
| F2 | Hβ | 25.7 | 50/50 | 0.0 | — | 48.7 | 78.5 | 0.9 |

*Mordenite

TABLE 2
Results on the evaluation of the catalyst activity

| Catalyst No. | | A1 | G1 | G2 | G3 | G4 | G5 | G6 | G7 | G8 |
|---|---|---|---|---|---|---|---|---|---|---|
| Supported | Mo | 4.0 | 4.0 | 4.0 | 4.0 | 4.0 | 4.0 | 4.0 | 4.0 | 4.0 |
| metal and | Co | — | 0.6 | — | — | — | — | 4.0 | — | 0.5 |
| its content | Ni | — | — | 1.2 | — | — | — | — | 0.8 | — |
| | Cr | — | — | — | 1.1 | — | — | — | — | 0.8 |
| | Zr | — | — | — | — | 0.8 | — | — | 1.0 | — |
| | Ag | — | — | — | — | — | 0.4 | — | — | 0.5 |

| | | | | | | | | | |
|---|---|---|---|---|---|---|---|---|---|
| Conversion of (Ben + C9 + A) | 62.6 | 63.3 | 62.7 | 63.1 | 62.7 | 62.1 | 65.3 | 63.5 | 65.0 |
| (wt %) | | | | | | | | | |
| Selectivity to (Tol + C8A) | 93.1 | 93.2 | 93.3 | 94.1 | 92.4 | 93.3 | 93.1 | 93.1 | 93.4 |
| (wt %) | | | | | | | | | |
| Tol selec./C8A select | 3.4 | 3.4 | 3.3 | 3.7 | 3.4 | 3.2 | 3.9 | 3.6 | 3.6 |
| (mole ratio) | | | | | | | | | |

TABLE 3
Results on the evaluation of the catalyst activity (I)

| | Ben/C₉A/C₁₀⁺A | Total | Selec. to | Tol selec./ |
|---|---|---|---|---|
| Catalyst | in feed | conversion | (Tol + C8A) | C8A select. |
| No. | (wt) | (wt %) | (wt %) | (mole) |
| G3 | 40/60/5 | 55.6 | 90.6 | 2.7 |
| G3 | 45/55/5 | 60.7 | 93.0 | 3.1 |
| G3 | 50/50/5 | 62.4 | 93.3 | 3.5 |
| G3 | 55/45/5 | 63.2 | 93.9 | 3.7 |
| G3 | 60/40/5 | 60.6 | 94.2 | 3.8 |

TABLE 4
Results on the evaluation of the catalyst activity (II)

| | | | | | | Selec. to | Tol |
|---|---|---|---|---|---|---|---|
| | React. | React. | | | Total | (Tol + | selec./C₈A |
| Catalyst | T | P | WHSV | H₂/HCₛ | conv. | C₈A) | select. |
| No. | (° C.) | (MPa) | (h⁻¹) | (mole) | (wt %) | (wt %) | (mole) |
| G3 | 300 | 3.0 | 2.5 | 5.3 | 42.8 | 72.4 | 1.8 |
| G3 | 350 | 3.0 | 2.5 | 5.3 | 57.4 | 88.7 | 2.8 |
| G3 | 400 | 3.0 | 2.5 | 5.3 | 64.6 | 91.4 | 3.7 |
| G3 | 480 | 3.0 | 2.5 | 5.3 | 62.4 | 84.6 | 4.0 |
| G3 | 380 | 2.0 | 2.5 | 5.3 | 55.3 | 89.6 | 2.7 |
| G3 | 380 | 2.5 | 2.5 | 5.3 | 60.6 | 91.8 | 3.1 |
| G3 | 380 | 3.0 | 2.5 | 5.3 | 63.1 | 94.1 | 3.7 |
| G3 | 380 | 4.0 | 2.5 | 5.3 | 63.3 | 93.3 | 3.8 |
| G3 | 380 | 3.0 | 1.0 | 5.3 | 63.9 | 93.6 | 4.0 |
| G3 | 380 | 3.0 | 2.0 | 5.3 | 63.5 | 93.8 | 3.8 |
| G3 | 380 | 3.0 | 3.0 | 5.3 | 62.9 | 94.2 | 3.6 |
| G3 | 380 | 3.0 | 4.0 | 5.3 | 55.1 | 94.4 | 2.8 |
| G3 | 380 | 3.0 | 5.0 | 5.3 | 50.6 | 94.7 | 2.1 |
| G3 | 380 | 3.0 | 2.5 | 2.5 | 51.3 | 74.1 | 1.7 |
| G3 | 380 | 3.0 | 2.5 | 4.0 | 56.7 | 85.6 | 2.1 |
| G3 | 380 | 3.0 | 2.5 | 5.5 | 63.2 | 94.4 | 3.7 |
| G3 | 380 | 3.0 | 2.5 | 7.0 | 65.4 | 94.6 | 3.8 |

TABLE 5
Stability test of the catalyst

| | | | | | Tol selec./ |
|---|---|---|---|---|---|
| | React. | React. T, | Average | Average selec. | C8A |
| Catalyst | Time | initial-end | conversion | to (Tol + C8A) | select. |
| No. | (h) | (° C.) | (wt %) | (wt %) | (mole) |
| A1 | 1000 | 380-380 | 62.2 | 93.0 | 3.2 |
| F1 | 500 | 410-445 | 48.0 | 84.5 | 1.2 |
| B1 | 1000 | 380-392 | 63.2 | 90.4 | 3.0 |
| F2 | 500 | 410-438 | 50.3 | 82.9 | 0.8 |

1. A transalkylation process of benzene and C₉⁺A for the production of toluene and C₈A comprising:
contacting a feed stream comprising benzene and C₉⁺A with a catalyst in a gas-solid phase fixed bed reactor, in the presence of hydrogen under the conditions of a benzene to C₉⁺A ratio of 10-90:90-10 by weight, a reaction temperature of 300-600□, a reaction pressure of 1.0-6.0 MPa, a weight hourly space velocity of the feed stream of 0.5-5.0 h⁻¹, and a hydrogen to hydrocarbon mole ratio of 1-15; based on the weight, the catalyst comprises 10-90 parts of a H-zeolite, 0.01-20 parts of molybdenum metal or/and oxide, and 10-90 parts of alumina; the H-zeolite has a SiO₂/Al₂O₃ mole ratio of 3-500.

2. The process of claim 1 wherein C₉⁺A is at least one selected from the group consisting of trimethylbenzene, methylethylbenzene, propylbenzene, indan, and C₁₀⁺ heavy aromatics.

3. The process of claim 1 wherein C₈ A is xylene, ethylbenzene or a mixture thereof.

4. The process of claim 1 wherein the molybdenum metal or/and oxide is 0.1-10.0 parts by weight in the catalyst.

5. The process of claim 1 wherein the molybdenum metal or/and oxide is 0.1-4.0 parts by weight in the catalyst.

6. The process of claim 1 wherein the SiO₂/Al₂O₃ mole ratio of the H-zeolite is 15-100.

7. The process of claim 1 wherein the SiO₂/Al₂O₃ mole ratio of the H-zeolite is 15-50.

8. The process of claim 1 wherein the H-zeolite is selected from the group consisting of ZSM-5 zeolite, Y-zeolite, mordenite, β-zeolite, MCM-22 zeolite, MCM-49 zeolite, MCM-56 zeolite, and combinations thereof.

9. The process of claim 1 wherein the H-zeolite is selected from the group consisting of mordenite, β-zeolite and combination thereof.

10. The process of claim 1, wherein the catalyst further comprises 0.01-20 parts by weight of an element in metal or/and oxide form selected from the group consisting of iron, cobalt, nickel, chromium, tungsten, bismuth, lanthanum, zirconium, silver, and combinations thereof.

11. The process of claim 10 wherein the element in metal or/and oxide form is 0.1-5.
